(19) United States
(12) Patent Application Publication
(10) Pub. No.: US 2025/0060349 A1
(43) Pub. Date: Feb. 20, 2025

(54) PESTICIDE RESIDUE EXTRACTION SOLUTION ADAPTED FOR RAPID EXTRACTION METHOD OF STOCK SOLUTION OF AGRICULTURAL PRODUCT AND METHOD FOR DETECTING AGRICULTURAL PRODUCT PESTICIDE RESIDUES USING THE PESTICIDE RESIDUE EXTRACTION SOLUTION THEREOF

(71) Applicant: Agricultural Chemicals Research Institute, Ministry of Agriculture, Taichung City (TW)

(72) Inventors: Shao-Kai LIN, Taichung City (TW); Wei-Chen CHUANG, Taichung City (TW)

(21) Appl. No.: 18/511,779

(22) Filed: Nov. 16, 2023

(30) Foreign Application Priority Data
Aug. 16, 2023 (TW) 112130854

(51) Int. Cl. G01N 33/24; G01N 1/40

(52) U.S. Cl. CPC G01N 33/24; G01N 1/4055; G01N 2001/4061; G01N 33/245

(57) ABSTRACT

A pesticide residue extraction solution adapted for a rapid extraction method of stock solution of agricultural products and a pesticide residue detection method in agricultural products. The pesticide residue extraction solution includes acidic solution and acetone solution. The rapid extraction method includes homogenizing the agricultural product sample, to obtain a homogenized sample; mixing the pesticide residue extraction solution with the homogenized sample to form a sample solution; and subsequently using a column filled with mixed powder to filter out the water and impurities from the sample solution, thereby obtaining a stock solution of the agricultural product. The method for detecting agricultural product pesticide residues includes using the rapid extraction method to obtain the stock solution of agricultural product, and to directly input the stock solution of the agricultural product into a liquid chromatography tandem mass spectrometer or a gas chromatography tandem mass spectrometer for pesticide residue detection.

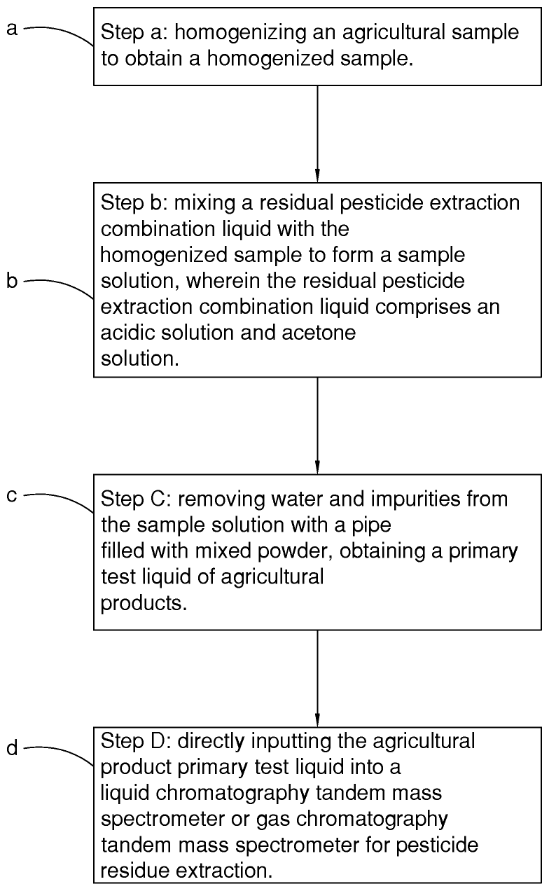

### FIELD OF THE INVENTION

The present invention involves technology related to the detection of pesticide residues in agricultural products, particularly to a residual pesticide extraction combination liquid for agricultural products, and to a rapid extraction method for obtaining primary test liquid from agricultural products using the residual pesticide extraction combination liquid, as well as a pesticide residue detection method in agricultural products.

### BACKGROUND OF THE INVENTION

For the detection of pesticide residues in agricultural products, liquid chromatography tandem mass spectrometry (LC/MS-MS) or gas chromatography tandem mass spectrometry (GC/MS-MS) instruments are commonly employed. Prior to using these instruments, a primary test liquid for instrument analysis is typically obtained from agricultural products using the QuEChERS method (Quick, Easy, Cheap, Effective, Rugged, and Safe). However, in recent years, there has been a growing trend towards the use of a rapid extraction kit for pesticide residue detection in agricultural products to enhance detection speed, with U.S. Pat. No. 9,581,579 being a typical example of such an innovation.

During the process of pesticide residue detection in agricultural products using the rapid extraction kit, it is necessary to add an extraction solvent to the homogenized agricultural sample to extract the primary test liquid. Currently, suitable extraction solvents are primarily acetonitrile solutions or acetonitrile solutions containing acetic acid. However, acetonitrile is a toxic chemical substance that poses risks to environmental pollution and human health. Another issue is that the primary test liquid extracted using solvents containing acetonitrile must undergo a solvent exchange step before it can be used for detection with Gas chromatography tandem mass spectrometer (GC/MS-MS). Therefore, addressing the aforementioned problems has been a long-standing concern in the pesticide detection industry.

### SUMMARY OF THE INVENTION

The present invention provides a residual pesticide extraction combination liquid suitable for agricultural products, as well as a rapid extraction method for obtaining primary test liquid from agricultural products using the residual pesticide extraction combination liquid, and a pesticide residue detection method in agricultural products. This addresses the issues associated with the use of acetonitrile solutions as extraction solvents in previous technologies, as mentioned above.

More specifically, the residual pesticide extraction combination liquid of the present invention comprises an acidic solution and acetone solution, wherein the content of the acidic solution is in the range of 0.5 to 3 vol %.

In one embodiment of the present invention, the content of the acidic solution in the residual pesticide extraction combination liquid is 1 vol %.

In one embodiment of the present invention, the acidic solution in the residual pesticide extraction combination liquid comprises formic acid, acetic acid, or a combination thereof.

The rapid extraction method for obtaining primary test liquid from agricultural products in the present invention comprises the following steps: homogenizing the agricultural sample to obtain a homogenized sample; mixing the residual pesticide extraction combination liquid described in the present invention with the homogenized sample to form a sample solution; and using a pipe filled with mixed powder to remove water and impurities from the sample solution, thereby obtaining a primary test liquid of agricultural products.

In one embodiment of the rapid extraction method for obtaining primary test liquid from agricultural products in the present invention, the agricultural sample used is vegetables or fruits, the homogenized sample is 1 gram, and the residual pesticide extraction combination liquid is 5 milliliters. Preferably, the rapid extraction method of the present invention further includes shaking the residual pesticide extraction combination liquid described in the present invention with the homogenized sample for 30 seconds to mix and form the sample solution.

In one embodiment of the rapid extraction method for obtaining primary test liquid from agricultural products in the present invention, deionized water is added to the homogenized sample, wherein the agricultural sample is grains or dried agricultural products. Preferably, the homogenized sample used in the rapid extraction method of the present invention is 0.5 grams, and the residual pesticide extraction combination liquid is 5 milliliters. More preferably, the rapid extraction method of the present invention further includes strongly shaking the residual pesticide extraction combination liquid with the homogenized sample for 30 seconds to mix and form the sample solution.

The pesticide residue detection method in agricultural products of the present invention comprises the following steps: obtaining an agricultural product primary test liquid using any one of the rapid extraction methods for obtaining primary test liquid from agricultural products as described in the present invention; and directly inputting the obtained agricultural product primary test liquid into a liquid chromatography tandem mass spectrometer or a gas chromatography tandem mass spectrometer for pesticide residue detection.

### BRIEF DESCRIPTION OF DRAWINGS

The FIGURE is a flow diagram of the pesticide residue detection method in accordance with the embodiment of the present invention.

### DETAILED DESCRIPTION OF THE INVENTION

The FIGURE depicts one embodiment of the pesticide residue detection method in agricultural products of the present invention, which includes the following steps:
Step a: homogenizing an agricultural sample to obtain a homogenized sample.
Step b: mixing a residual pesticide extraction combination liquid with the homogenized sample to form a sample solution, wherein the residual pesticide extraction combination liquid comprises an acidic solution and acetone solution, and the content of the acidic solution is in the range of 0.5 to 3 vol % and more preferably 1 vol %. The acidic solution can be formic acid, acetic acid, or a combination thereof, but is not limited to these. Additionally, in another embodiment, the residual pesticide extraction combination liquid consists only of the acidic solution and acetone solution, without any other components.
Step c: removing water and impurities from the sample solution with a pipe filled with mixed powder, obtaining a primary test liquid of agricultural products. In this embodiment, the rapid extraction kit from U.S. Pat. No. 9,581,579 can be optionally chosen as the pipe filled with mixed powder, but it is not limited to this option.
Step d: directly inputting the agricultural product primary test liquid into a liquid chromatography tandem mass spectrometer or gas chromatography tandem mass spectrometer for pesticide residue extraction. For example, inputting 1 ml of the agricultural product primary test liquid into the mentioned instrument for pesticide residue detection.

In step a, the agricultural sample can be fresh vegetables or fruits, as well as dried grains or other dried agricultural products, but is not limited to the aforementioned options. In this embodiment, the agricultural sample is homogenized into a powdered homogenized sample using a blender.

In step b, the residual pesticide extraction combination liquid and the homogenized sample are strongly shaken together for a period of time to ensure thorough mixing and the formation of a uniform sample solution. In this embodiment, the agricultural sample is fresh vegetable or fruit, the homogenized sample is 1 gram, the residual pesticide extraction combination liquid is 5 ml, and the shaking time is 30 seconds, but it is not limited to these specific values.

In another embodiment, the agricultural sample is dried grains or other dried agricultural products, and the homogenized sample needs to have proper quantity of deionized water added to it, and stand for a suitable period, for example, 10 minutes. Subsequently, the homogenized sample with added deionized water is strongly shaken together with the residual pesticide extraction combination liquid for a certain period to ensure thorough mixing and the formation of a uniform sample solution.

In step c, the sample solution is poured into the pipe, and then the sample solution is pressurized with a piston rod to make it flow through the mixed powder in the pipe at a predetermined flow rate. The preferred predetermined flow rate is in the range of 0.01 to 0.2 ml/sec, with 0.05 ml/sec being more preferable. Additionally, the air exhausting method can also be used to drive the sample solution to flow through the mixed powder in the pipe at the predetermined flow rate. The mixed powder can absorb some of the water content and impurities in the sample solution, especially those impurities that may interfere with the results of the aforementioned instruments.

The time required to press the sample solution using the piston rod is typically less than 1 minute. In the above-mentioned embodiment, the sample solution drips out from the pipe at a rate of one drop per second, and the time required for pressing the sample solution is approximately 30 seconds. In other words, after pressing for approximately 30 seconds, the entire sample solution has been filtered by the mixed powder and is output from the pipe to form the primary test liquid of agricultural products.

In step d, 1 ml of the primary test liquid of agricultural products is taken and input into the aforementioned instrument, which then detects the pesticide residues in the agricultural product primary test liquid. Furthermore, one embodiment of the rapid extraction method for obtaining primary test liquid from agricultural products of the present invention includes the steps from a to c, without going into further detail.

TABLE 1

| | | Average pesticide | Average pesticide |
|---|---|---|---|
| | | recovery rate of | recovery rate of |
| | | the control | the present invention |
| No. | Name of Pesticides | method (%) | method (%) |
| 1 | Abamectin | 75.0 | 73.6 |
| 2 | Acephate | 75.0 | 86.1 |
| 3 | Acetamiprid | 84.7 | 94.3 |
| 4 | Acrinathrin | 88.0 | 121.5 |
| 5 | Alachlor | 96.3 | 97.5 |
| 6 | Ametoctradin | 87.3 | 97.2 |
| 7 | Ametryn | 86.6 | 91.1 |
| 8 | Amisulbrom | 99.5 | 113.8 |
| 9 | Atrazine | 96.2 | 94.3 |
| 10 | Azinphos-methyl | 103.6 | 90.9 |
| 11 | Azoxystrobin | 95.2 | 102.6 |
| 12 | Bendiocarb | 98.4 | 101.0 |
| 13 | Benfuracarb | 34.3 | 87.4 |
| 14 | Bensulfuron-methyl | 150.2 | 95.2 |
| 15 | Bifenazate | 65.6 | 82.6 |
| 16 | Bifenthrin | 69.8 | 89.9 |
| 17 | Bitertanol | 114.6 | 105.9 |
| 18 | Boscalid | 98.5 | 96.0 |
| 19 | Bromacil | 95.7 | 106.8 |
| 20 | Bromopropylate | 133.9 | 122.8 |
| 21 | Bromuconazole | 107.6 | 100.6 |
| 22 | Bupirimate | 91.2 | 109.1 |
| 23 | Buprofezin | 79.0 | 89.3 |
| 24 | Butachlor | 104.0 | 96.2 |
| 25 | Butralin | 76.4 | 91.7 |
| 26 | Carbaryl | 100.0 | 94.7 |
| 27 | Carbendazim | 61.6 | 88.5 |
| 28 | Carbofuran | 98.0 | 97.5 |
| 29 | Chlorantraniliprole | 105.6 | 94.5 |
| 30 | Chlorfenapyr | ND | 108.1 |
| 31 | Chlorfluazuron | 71.5 | 103.3 |
| 32 | Chlorothalonil | 54.8 | 11.2 |
| 33 | Chlorpropham | 137.0 | ND |
| 34 | Chlorpyrifos | 87.8 | 99.6 |
| 35 | Chromafenozide | 91.2 | 90.4 |
| 36 | Clofentezine | 87.4 | 93.9 |
| 37 | Clothianidin | 100.1 | 117.0 |
| 38 | Cyantraniliprole | 89.3 | 85.7 |
| 39 | Cyazofamid | 93.5 | 93.5 |
| 40 | Cyenopyrafen | 49.8 | 58.8 |
| 41 | Cyflufenamid | 102.4 | 101.7 |
| 42 | Cyflumetofen | 103.7 | 94.9 |
| 43 | Cyfluthrin | ND | ND |
| 44 | Cyhalothrin | 157.3 | 144.4 |
| 45 | Cymoxanil | 107.8 | 100.0 |
| 46 | Cypermethrin | 99.3 | 68.4 |
| 47 | Cyproconazole | 111.5 | 105.4 |
| 48 | Cyprodinil | 77.4 | 99.9 |
| 49 | Cyromazine | ND | ND |
| 50 | Deltamethrin | 92.7 | 85.1 |
| 51 | Diazinon | 93.2 | 87.9 |
| 52 | Difenoconazole | 83.7 | 91.8 |
| 53 | Diflubenzuron | 99.1 | 103.9 |
| 54 | Dimethoate | 82.9 | 96.3 |
| 55 | Dimethomorph | 109.9 | 106.9 |
| 56 | Diniconazole | 96.7 | 96.4 |
| 57 | Dinotefuran | 51.0 | 85.4 |
| 58 | Diuron | 90.5 | 97.6 |
| 59 | Edifenphos | 104.7 | 106.7 |
| 60 | Emamectin benzoate | 73.2 | 105.0 |
| | B1a | | |
| 61 | Epoxiconazole | 90.8 | 106.6 |
| 62 | Ethion | 82.2 | 89.2 |
| 63 | Ethiprole | 97.7 | 120.1 |
| 64 | Ethirimol | 84.4 | 77.9 |
| 65 | Etofenprox | 80.0 | 93.4 |
| 66 | Etoxazole | 79.2 | 91.1 |
| 67 | Etridiazole | ND | ND |
| 68 | Famoxadone | 79.2 | 95.1 |
| 69 | Fenamiphos | 93.4 | 86.8 |
| 70 | Fenarimol | 103.5 | 95.8 |
| 71 | Fenazaquin | 81.7 | 90.8 |
| 72 | Fenbuconazole | 100.3 | 97.9 |
| 73 | Fenbutatin-oxide | 75.2 | 75.9 |
| 74 | Fenitrothion | 168.5 | ND |
| 75 | Fenobucarb | 94.2 | 90.8 |
| 76 | Fenothiocarb | 102.7 | 98.8 |
| 77 | Fenoxanil | 102.1 | 90.5 |
| 78 | Fenoxycarb | 90.7 | 87.9 |
| 79 | Fenpropathrin | 79.4 | 74.1 |
| 80 | Fenpyroximate | 81.8 | 79.4 |
| 81 | Fenthion | 102.7 | 110.5 |
| 82 | Fenvalerate | ND | ND |
| 83 | Fipronil | 105.1 | 93.4 |
| 84 | Fipronil-sulfone | 94.5 | 90.2 |
| 85 | Flonicamid | 64.5 | 85.1 |
| 86 | Fluazinam | 68.8 | 71.7 |
| 87 | Flubendiamide | 96.6 | 106.5 |
| 88 | Flucythrinate | 97.0 | 88.7 |
| 89 | Fludioxonil | 94.1 | 97.4 |
| 90 | Fluensulfone | ND | ND |
| 91 | Flufenoxuron | 83.2 | 95.5 |
| 92 | Fluopicolide | 106.5 | 117.4 |
| 93 | Fluopyram | 91.0 | 97.3 |
| 94 | Flupyradifurone | 104.5 | 99.2 |
| 95 | Flusilazole | 93.2 | 99.9 |
| 96 | Flutolanil | 96.9 | 108.0 |
| 97 | Flutriafol | 95.5 | 103.3 |
| 98 | Fluvalinate | 101.3 | 94.6 |
| 99 | Fluxapyroxad | 122.3 | 126.7 |
| 100 | Formetanate | 74.2 | 78.1 |
| 101 | Fosthiazate | 91.5 | 88.7 |
| 102 | Hexaconazole | 101.0 | 100.0 |
| 103 | Hexythiazox | 73.0 | 97.9 |
| 104 | Imibenconazole | 64.3 | 85.6 |
| 105 | Imidacloprid | 105.1 | 107.8 |
| 106 | Indoxacarb | 93.9 | 88.6 |
| 107 | Iprobenfos | 95.6 | 90.2 |
| 108 | Iprodione | 100.4 | 112.6 |
| 109 | Isoprocarb | 92.9 | 92.3 |
| 110 | Isoprothiolane | 105.1 | 99.1 |
| 111 | Isopyrazam | 97.0 | 97.1 |
| 112 | Isoxathion | 98.3 | 104.7 |
| 113 | Kresoxim-methyl | 82.6 | 82.9 |
| 114 | Linuron | 96.8 | 98.7 |
| 115 | Lufenuron | 87.4 | 106.0 |
| 116 | Malathion | 93.7 | 94.2 |
| 117 | Mandipropamid | 101.8 | 107.4 |
| 118 | Mefenacet | 92.7 | 102.6 |
| 119 | Mepanipyrim | 83.2 | 103.5 |
| 120 | Mepronil | 96.4 | 96.0 |
| 121 | Metaflumizone | 67.5 | 60.7 |
| 122 | Metalaxyl | 99.5 | 92.4 |
| 123 | Metconazole | 92.3 | 95.4 |
| 124 | Methamidophos | 71.9 | 90.9 |
| 125 | Methidathion | 89.9 | 89.4 |
| 126 | Methiocarb | 98.6 | 95.7 |
| 127 | Methomyl | 101.6 | 111.0 |
| 128 | Methoxyfenozide | 96.4 | 98.9 |
| 129 | Metolachlor | 101.3 | 111.9 |
| 130 | Metrafenone | 99.4 | 101.2 |
| 131 | Metribuzin | 104.4 | 103.5 |
| 132 | Monocrotophos | 79.4 | 92.7 |
| 133 | Myclobutanil | 94.5 | 84.9 |
| 134 | Napropamide | 84.9 | 100.8 |
| 135 | Nitenpyram | 105.4 | 88.8 |
| 136 | Novaluron | 82.0 | 87.1 |
| 137 | Omethoate | 68.8 | 89.0 |
| 138 | Oxadiazon | 110.0 | 122.1 |
| 139 | Oxadixyl | 90.2 | 96.1 |
| 140 | Oxamyl | 73.8 | 94.6 |
| 141 | Oxycarboxin | 115.3 | 100.2 |
| 142 | Paclobutrazol | 95.5 | 93.5 |
| 143 | Penconazole | 105.1 | 99.3 |
| 144 | Pencycuron | 102.6 | 107.4 |
| 145 | Pendimethalin | 93.4 | 85.9 |
| 146 | Permethrin | 61.3 | 97.0 |
| 147 | Phenthoate | 81.0 | 107.1 |
| 148 | Phorate | 86.6 | 82.9 |
| 149 | Phosmet | 103.5 | 94.4 |
| 150 | Piperonyl butoxide | 86.8 | 92.5 |
| 151 | Probenazole | 98.6 | 101.3 |
| 152 | Prochloraz | 85.7 | 93.4 |
| 153 | Procymidione | 149.5 | 97.0 |
| 154 | Profenophos | 78.7 | 99.3 |
| 155 | Propamocarb | 81.3 | 140.1 |
| | hydrochloride | | |
| 156 | Propargite | 103.6 | 94.5 |
| 157 | Propiconazole | 88.3 | 88.1 |
| 158 | Propoxur | 91.1 | 92.4 |
| 159 | Proquinazid | 75.9 | 87.3 |
| 160 | Prothiofos | 82.5 | 90.4 |
| 161 | Pymetrozine | 47.9 | ND |
| 162 | Pyraclofos | 92.6 | 100.7 |
| 163 | Pyraclostrobin | 86.1 | 96.1 |
| 164 | Pyridaben | 100.4 | 114.2 |
| 165 | Pyrimethanil | 83.0 | 98.7 |
| 166 | Pyrimidifen | 78.4 | 87.8 |
| 167 | Pyriproxyfen | 80.1 | 104.9 |
| 168 | Quinoxyfen | 85.1 | 103.6 |
| 169 | Rotenone | 91.6 | 104.2 |
| 170 | SpinetoramJ | 86.2 | 93.1 |
| 171 | SpinetoramL | 79.5 | 89.7 |
| 172 | SpinosadA(spinosinA) | 88.3 | 86.9 |
| 173 | SpinosadD(spinosinD) | 79.0 | 83.7 |
| 174 | Spirodiclofen | 83.9 | 89.6 |
| 175 | Spiromesifen | 97.1 | 113.1 |
| 176 | Spirotetramat | 87.6 | 90.6 |
| 177 | Sulfoxaflor | 93.6 | 102.9 |
| 178 | Tebuconazole | 124.1 | 108.6 |
| 179 | Tebufenozide | 97.0 | 86.0 |
| 180 | Tebufenpyrad | 88.9 | 91.9 |
| 181 | Terbufos | 81.9 | 84.3 |
| 182 | Tetraconazole | 89.3 | 82.9 |
| 183 | Thiabendazole | 61.7 | 86.9 |
| 184 | Thiacloprid | 97.4 | 98.5 |
| 185 | Thiamethoxam | 91.8 | 83.0 |
| 186 | Thifluzamide | 77.9 | 108.8 |
| 187 | Thiobencarb | 95.9 | 99.6 |
| 188 | Thiodicarb | 54.3 | 72.6 |
| 189 | Tolclofos-methyl | 154.0 | 105.5 |
| 190 | Tolfenpyrad | 89.3 | 99.1 |
| 191 | Triadimefon | 89.2 | 106.1 |
| 192 | Triadimenol | 108.0 | 96.7 |
| 193 | Triazophos | 98.9 | 102.9 |
| 194 | Trichlorfon | 122.8 | 124.1 |
| 195 | Tricyclazole | 62.1 | 81.8 |
| 196 | Trifloxystrobin | 83.9 | 86.2 |
| 197 | Triflumizole | 88.8 | 91.7 |
| 198 | Zoxamide | 97.9 | 104.1 |

Table 1 presents the results of pesticide residue detection in an agricultural product sample using the method of the present invention compared to a control method. The agricultural product sample is Qingjiang cabbage, and it contains 198 known concentrations of pesticide standards. These pesticide standards include acaricides (such as Clofentezine and Ethion), fungicides (such as Iprobenfos and Penconazole), herbicides (such as bromacil and Alachlor), insecticides (such as Fenobucarb and Diflubenzuron), nematicides (such as Fenamiphos and Fosthiazate), plant growth regulators (such as Paclobutrazol), and other pesticides (such as Piperonyl butoxide), as detailed in Table 1, without going into further detail.

In Table 1, the extraction solvent used in the method of the present invention is the residual pesticide extraction combination liquid, with a 1 vol % of acetic acid, and the rest being acetone solution. The steps performed in the control method are generally similar to those in the present invention, with the key difference that the extraction solvent used in the control method is a mixture of acetic acid and acetone solution, with a 1 vol % content of acetic acid and the rest being acetone solution. Additionally, “ND” in Table 1 and the following Table 2 refers to values where the peak signal-to-noise ratio (PSNR) is less than 10, and recovery rates are not calculated for these cases.

Table 2 presents the statistical results based on the data from Table 1.

TABLE 2

| | | Method of the |
|---|---|---|
| | Control method | present invention |
| | Number of | Number of |
| | pesticides | pesticides |
| | falling within the | falling within the |
| | average recovery | average recovery |
| Average Recovery Range(%) | range | range |
| ND | 6 | 8 |
| Less than or equal to 50% | 3 | 1 |
| Greater than 50% and less | 3 | 1 |
| than or equal to 60% | | |
| Greater than 60% and less | 11 | 2 |
| than 70% | | |
| Greater than 70% and less | 165 | 178 |
| than or equal to 120% | | |
| Greater than 120% and less | 5 | 6 |
| than or equal to 140% | | |
| Greater than 140% | 5 | 2 |
| The total number of pesticides | 198 | 198 |

Acording to Table 2, the recovery rates for as many as 178 pesticides using the present invention's method fall within the standard range of greater than or equal to 70% and less than or equal to 120%. In contrast, the control method only achieves recovery rates within this range for 165 pesticides. Furthermore, the recovery rate of one pesticide in the present invention is less than or equal to 50%, and the recovery rate of another pesticide falls within the range of greater than 50% and less than or equal to 60%. The present invention also has two pesticides with recovery rates falling within the range of greater than 60% and less than or equal to 70%. Additionally, there are six pesticides with recovery rates falling within the range of greater than 120% and less than or equal to 140%, and two pesticides with recovery rates exceeding 140%. Overall, the present invention's method outperforms the control method in terms of pesticide recovery rates. Furthermore, because the present invention's method in step b uses a residual pesticide extraction combination liquid that does not contain ethion, the agricultural product primary test liquid can be directly input into the aforementioned instruments without the need for solvent replacement. Additionally, since the present invention's method does not employ the use of the toxic chemical ethion, there is no risk of environmental contamination or harm to human health.

1. A method for rapidly extracting primary test liquid from agricultural products, comprising:
homogenizing the agricultural sample to obtain a homogenized sample;
mixing a residual pesticide extraction combination liquid with the homogenized sample to form a sample solution, wherein the residual pesticide extraction combination liquid comprises an acidic solution and acetone solution, and the content of the acidic solution is in the range of 0.5 to 3 vol %; and
removing water and impurities from the sample solution by an pipe filled with mixed powder, obtaining a primary test liquid of agricultural products.

2. The method as recited in claim 1, wherein the acidic solution has a content of 1 vol %.

3. The method as recited in claim 1, wherein the acidic solution consists of formic acid, acetic acid, or a combination thereof.

4. The method as recited in claim 1, wherein the agricultural sample is vegetables or fruits, the homogenized sample is 1 gram, and the residual pesticide extraction combination liquid is 5 ml.

5. The method as recited in claim 4, wherein the residual pesticide extraction combination liquid is shaken together with the homogenized sample for 30 seconds to mix the two and form the sample solution.

6. The method as recited in claim 4, comprising adding deionized water to the homogenized sample, wherein the agricultural sample is grains or dried agricultural products.

7. The method as recited in claim 6, wherein the homogenized sample is 0.5 grams, the residual pesticide extraction combination liquid is 5 ml, and the deionized water is 1 milliliter.

8. The method as recited in claim 7, wherein the residual pesticide extraction combination liquid is shaken together with the homogenized sample for 30 seconds to mix the two and form the sample solution.

9. A pesticide residue detection method, comprising:
obtaining an agricultural product primary test liquid using the rapid extraction method recited in claim 1; and
directly inputting the agricultural product primary test liquid into a Liquid chromatography tandem mass spectrometer or Gas chromatography tandem mass spectrometer for pesticide residue extraction.

10. A residual pesticide extraction combination liquid for agricultural products, comprising an acidic solution and acetone solution, wherein the content of the acidic solution is in the range of 0.5 to 3 vol %.

11. The residual pesticide extraction combination liquid as recited in claim 1, wherein the content of the acid solution is 1 vol %.

12. The residual pesticide extraction combination liquid as recited in claim 10, wherein the acidic solution comprises formic acid, acetic acid, or a combination thereof.

13. The residual pesticide extraction combination liquid as recited in claim 11, wherein the acidic solution comprises formic acid, acetic acid, or a combination thereof.
